(19) United States
(12) Patent Application Publication
(10) Pub. No.: US 2021/0247393 A1
(43) Pub. Date: Aug. 12, 2021

(54) SYSTEM AND METHOD OF TESTING VETERINARY HEALTH

(71) Applicant: PetDx, Inc., La Jolla, CA (US)

(72) Inventors: Ilya Chorny, La Jolla, CA (US); Nilesh Dharajiya, La Jolla, CA (US); Daniel Grosu, La Jolla, CA (US); Arthur Polk, La Jolla, CA (US)

(21) Appl. No.: 17/262,159

(22) PCT Filed: Sep. 30, 2020
(86) PCT No.: PCT/US2020/053437

§ 371 Date: Jan. 21, 2021

Related U.S. Application Data
(60) Provisional application No. 62/909,625, filed on Oct. 2, 2019.

(51) Int. Cl. G01N 33/564; G16H 10/60; G16H 40/40

(52) U.S. Cl. CPC G01N 33/564; G16H 40/40; G16H 10/60

(57) ABSTRACT

The systems and methods herein aid in coordinating the activities of disparate entities for assessing the status of cancer or other diseases in pets. A streamlined process by the systems and methods herein allow consumers to expedite the logistics for receiving medical kits, setting up an appointment with a clinician, sending a sample associated with medical tests to a testing lab, and assessing the health of the pet based on the sample. By coordinating these activities by the systems and methods herein, earlier stage diagnosis of the consumer's pet can be achieved.

### INCORPORATION BY REFERENCE TO RELATED APPLICATIONS

This application is a U.S. National Phase of PCT Application No. PCT/US2020/053437, filed Sep. 30, 2020, which designates the United States of America, and published in the English language, and which is an International Application of and claims the benefit of priority to U.S. Provisional Application No. 62/909,625, filed Oct. 2, 2019. The disclosures of the above-referenced applications are hereby incorporated by reference in their entireties.

### FIELD

The invention pertains to the field of logistics management. More particularly, the invention pertains to the expedited delivery of medical tests for assessing and detecting cancer and other disease states in pets by coordinating activities between disparate entities.

### BACKGROUND

Pets are animals that can be an integral part of a human's life by providing the human with a sense of companionship and affection. To this end, humans can treat pets as members of their family and thus require adequate healthcare for the well-being of the pets. Common pets include cats and dogs. Some other pets include lizards, hamsters, and gerbils.

Dogs and humans have a similar lifetime prevalence of cancer. However, dogs have a much shorter life span than humans. As a result, the incidence of cancer in dogs is more than ten times higher than in humans, on an annual basis. In the United States, the population of humans is approximately 4 times larger than the population of dogs (330 million vs. 90 million, respectively. Yet, the number of new cancer diagnoses for dogs is approximately 4 times higher than for humans (6 million vs. 1.6 million, respectively). Despite the biology of cancer in dogs and humans being similar, the development of cancer medical tests and therapeutics for dogs has lagged behind the development of cancer medical tests and therapeutics for humans. Early detection of cancer or other diseases can aid in maintaining the health and quality of life, and extending the span of life, of dogs or other pets.

### SUMMARY

The present teachings include a computer implemented method for assessing a health state of canines or other animals. The method can include: receiving an order from an individual consumer for a medical test without the need for a prescription and thereby shipping a medical testing kit to the individual consumer or a clinician, wherein the medical testing kit contains one or more items for supporting the medical test; confirming receipt of the medical testing kit by the individual consumer; sending instructions to collect a sample for the medical testing kit; shipping the medical testing kit containing the sample to a medical testing laboratory, wherein the sample is stored in an item of the one or more items and the sample derives from the canines or other animals; confirming receipt of the sample in the medical testing kit by the medical testing laboratory; making an assessment of the health state based on criteria applied on the sample in the medical test kit; and sending a prompt to the clinician, wherein the prompt triggers an interface for modifying the assessment.

In accordance with a further aspect, the computer implemented method can include: receiving a profile of the sample.

In accordance with a further aspect of the computer implemented method, the shipping the medical testing kit containing the sample is optionally performed by the clinician.

In accordance with a further aspect of the computer implemented method, the shipping the medical testing kit containing the sample can include: receiving a verification of the profile of the sample prior to shipping the sample by the clinician.

In accordance with a further aspect of the computer implemented method, the profile can be processed electronically.

In accordance with a further aspect of the computer implemented method, the medical test can be a liquid biopsy test for detection of cancer.

In accordance with a further aspect of the computer implemented method, the assessment of the health state can be sent to a GUI by the clinician prior to sending the assessment of the health state to the individual consumer.

In accordance with a further aspect of the computer implemented method, the sample can be blood.

In accordance with a further aspect of the computer implemented method, the medical testing kit can contain a blood tube designed to stabilize ctDNA for liquid biopsy applications.

In accordance with yet another aspect, the computer implemented method can include: generating a list sent to the individual consumer, wherein the list contains clinicians for selection by the individual consumer, and wherein a clinician selected from the list collects the sample for the medical testing kit and places the sample in the medical testing kit.

In accordance with yet another aspect, the computer implemented method can include: sending the assessment of the health state to the individual consumer or to a GUI as a PDF.

The present teachings include a computer implemented system for ordering a medical test for canines or other animals by an individual consumer. The computer implemented system can include a server and a computing device in communication with the server over a network. The computer device can include a memory coupled to the processor and configured to provide the processor with instructions for: receiving order directly from the individual consumer without the need for a prescription for the medical test and thereby shipping the medical testing kit to the individual consumer or a clinician or the individual consumer and the clinician, wherein the medical testing kit contains one or more items for supporting the medical test; sending a request to a logistics organization to ship the medical kit to the individual consumer; receiving a confirmation of receipt that the individual consumer received the medical kit; receiving a request from the individual consumer to select the clinician to collect a sample for the medical testing kit; sending a notification of receipt of the sample by a testing laboratory to the individual consumer, wherein the sample is stored in an item of the one or more times and the sample derives from the canines or other animals; sending a notification of the availability of a diagnosis obtained from the medical test of the sample to the individual consumer; making an assessment of a health state of the canines or other animals, based on criteria applied on the sample in the medical testing kit; sending a notification of the assessment of the health state to the individual consumer; and sending a prompt to the clinician, wherein the prompt triggers an interface for optionally modifying the assessment.

In accordance with yet another aspect of the computer implemented system, the medical test can be a liquid biopsy test for the detection of cancer.

In accordance with yet another aspect, the computer implemented system can include instructions for: generating a list sent to the individual consumer, wherein the list contains clinicians by the individual consumer, and wherein a clinician selected from the list collects the sample for the medical testing kit and places the sample in the medical testing kit; receiving a request from the individual consumer to schedule an appointment with the selected clinician; and sending a notification of confirmation that the appointment has been scheduled with the selected clinician.

In accordance with a further aspect of the computer implemented system, the sample can be blood.

In accordance with a further aspect of the computer implemented system, the sample test kit can contain a blood tube designed to stabilize ctDNA for liquid biopsy applications.

In accordance with a further aspect, the computer implemented system can involve instructions for: receiving a profile of the sample.

The present teachings include a computer program product for ordering a medical test for canines or other animals by an individual consumer. The computer program product can include a server and a computing device in communication with the server over a network. The computer device can be in communication with the server over a network. The computing device can include a processor configured to provide the processor with instructions configured to provide the processor with instructions for: receiving an order directly from the individual consumer without the need for a prescription for the medical test and thereby shipping the medical testing kit to the individual consumer or a clinician or the individual consumer and the clinician, wherein the medical testing kit contains one or more items for supporting the medical test; sending a request to a logistics organization to ship the medical kit to the individual consumer; receiving a confirmation of receipt that the individual consumer received the medical kit; receiving a request from the individual consumer to select the clinician to collect a sample for the medical testing kit; sending a notification of receipt of the sample by a testing laboratory to the individual consumer, wherein the sample is stored in an item of the one or more times and the sample derives from the canines or other animals; sending a notification of the availability of a diagnosis obtained from the medical test of the sample to the individual consumer; making an assessment of a health state of the canines or other animals, based on criteria applied on the sample in the medical testing kit; sending a notification of the assessment of the health state to the individual consumer; and sending a prompt to the clinician, wherein the prompt triggers an interface for optionally modifying the assessment.

In accordance with yet another aspect of the computer program product, the medical test can be a liquid biopsy test for the detection of cancer.

In accordance with yet another aspect, the computer program product can include instructions for: generating a list sent to the individual consumer, wherein the list contains a clinician for selection by the individual consumer, and wherein a clinician selected from the list collects the sample for the medical testing kit and places the sample in the medical testing kit; receiving a request from the individual consumer to schedule an appointment with the selected clinician; and sending a notification of confirmation that the appointment has been scheduled with the selected clinician.

In accordance with a further aspect of the computer implemented system, the sample can be blood.

In accordance with a further aspect of the computer program product, the sample test kit can contain a blood tube designed to stabilize ctDNA for liquid biopsy applications.

In accordance with a further aspect, the computer program product can involve instructions for: receiving a profile of the sample.

These and other features, aspects, and advantages of the present teachings will become better understood with reference to the following description, examples and appended claims.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a block diagram that depicts a computing environment for the expedited and streamlined delivery of medical tests for assessing and detecting disease states in pets by coordinating activities among disparate entities.

FIG. 2A, FIG. 2B, FIG. 2C, and FIG. 2D depict interfaces generated by the server operated by the coordination entity.

FIG. 2E is a perspective view of one embodiment of a virtual medical testing kit for veterinary health.

FIG. 3 depicts a flowchart for the expedited and streamlined delivery of medical tests for assessing and detecting cancer and disease states in pets by coordinating activities among disparate entities.

FIG. 4A, FIG. 4B, FIG. 4C, FIG. 4D, FIG. 4E, FIG. 4F, and FIG. 4G depict interfaces for modifying profile information of the pets and the report assessing the health of the pet.

FIG. 5A, FIG. 5B, and FIG. 5C depict interfaces with the consumer entities that support customized delivery of medical testing kits and tele-health delivery of reports assessing the health of the pet.

FIG. 6A depicts a computer system having a control unit, filter techniques, and a database for implementing one embodiment of the systems and methods herein.

FIG. 6B depicts a computer system having a control unit and AI techniques for determining a result according to one embodiment.

### DETAILED DESCRIPTION

Embodiments of the invention relate to systems and methods for delivering medical tests, particularly for veterinary health. Systems and methods herein may be used for early detection of cancer or other diseases in pets. More specifically, the delivery of medical test by the systems and methods herein may aid in early detection of cancer or other diseases in pets. Detection of cancer in the early stages may lead to better outcomes than detection of cancer in the later stages.

One embodiment is a system or method that allows a consumer to manage the ordering and processing of a genetic medical test for their animal. By going on-line, the consumer can fill out the proper information and order a genetic medical testing kit that can be shipped directly to their home or office. The systems and methods herein may then track and manage the testing process for the consumer. The test kit can be delivered to or picked up at a veterinarian of the consumer's choosing. In one embodiment, the systems and methods herein may give the consumer options for veterinarians, based on the geographic location of veterinarians in relation to the consumer and clinical practice factors. Clinical practice factors include, but are not limited to, at least one of: (i) veterinarians who stock the genetic medical testing kit; (ii) veterinarians have prior experience obtaining samples for the genetic medical testing kit; and (iii) wait times at the veterinarians” offices. Once the consumer has access to the test kit and brings the test kit to the selected veterinarian's office, the selected veterinarian can draw blood from the animal in a simple procedure and then ship the blood sample back to a testing lab using the medical testing kit. The medical testing kits can contain typical tubes, needles, caps, and stabilizing agents configured to store and ship blood to a testing lab or facility.

Once the tube of animal blood has been received by the testing lab, it can be tested for a variety of genetic diseases, including cancer, by using liquid biopsy techniques. For example, a circulating tumor DNA (ctDNA) analysis may be performed on the blood to determine if any ctDNA is found in the blood. If ctDNA is found, the lab may identify the type and stage of the cancer through analysis of the ctDNA in the blood. Once the lab has results of their tests, those results can be entered into the system.

Once the testing has been completed, the veterinarian or the consumer may receive a notification that the testing is complete and the veterinarian and consumer can log into a website or portal to review their results. If the results indicate that the animal has cancer, further information regarding the stage of cancer and recommendations for local veterinarians who specialize in cancer treatment may also be presented to the consumer.

In one embodiment, the systems and methods herein detect cancer using a liquid biopsy that screens for ctDNA in the blood. In stage I cancer, at least 40% of the cases are estimated to be detectable with ctDNA and the estimated 10-year survival rate is 90%. In stage II cancer, at least 50% of the cases are estimated to be detectable with ctDNA and the estimated 10-year survival rate is 70%. In stage III cancer, at least 63% of the cases are estimated to be detectable with ctDNA and the estimated 10-year survival rate is 38%. Finally, in stage IV cancer, at least 77% of the cases are detectable with ctDNA and the estimated 10-year survival rate is 5%. These statistics, which are approximations derived from experience in humans, directionally translate to veterinary medicine as well. In fact, pets are usually diagnosed with cancer at later stages compared to humans because animals are: (i) known to be more pain tolerant than humans and (ii) unable to communicate that something is negatively impacting them. As a result, pets have a short life expectancy post diagnosis.

In the early stages, a cure can be achieved more readily by surgery and chemotherapy as the cancer is more localized and has not spread to the lymphatics or to distant organs. Early detection is possible by using assays that test for blood biomarkers, such as ctDNA. The systems and methods herein support a direct-to-consumer distribution channel that allows consumers to be directly involved in their animal's health decisions. The direct-to-consumer distribution channel herein coordinates activities between disparate entities. The disparate entities include: the humans owning the pet, pets, veterinarians, pathologists, medical testing laboratories, and companies for transporting medical testing kits to the appropriate entity. More specifically, the direct-to-consumer distribution channel connects a consumer entity (i.e., humans owning the pets) to the appropriate clinical entities (i.e., veterinarians), testing entities (i.e., pathologists and medical testing laboratories), and logistic entities (i.e., companies for transporting medical testing kits to the appropriate entity). Additionally, the direct-to-consumer distribution channel herein enhances the probability of earlier cancer detection by: (i) delivering the medical testing kit to the human as a kit without the need for a prescription; (ii) identifying and facilitating a veterinarian or equivalent entity trained in collecting a sample for the medical test ; (iii) sending the sample to a medical testing laboratory; (iv) assessing the health of the pet based on the analysis of the sample; (v) streamlining and integrating payment processing; (vi) facilitating dog owners personal and health information security; and (vii) validating pet profile through several interactions to increase clinical integrity. Furthermore, if the health of the pet is assessed to be at risk for cancer, or as having cancer, a treatment regimen can be devised for the pet by identifying a veterinarian (or another veterinarian) to assist in the treatment. This treatment regimen can result in a cure or in a life extension for the pet assed to be at risk for cancer or has cancer.

In one embodiment, the systems and methods described herein include: (i) allowing a pet owner (i.e., the consumer entity) to order a blood testing kit online and pay for a blood collection at the same time without the need for a prescription; and (ii) providing unique convenience and streamlining the medical testing process for a pet. More specifically, the consumer entity is in charge of ordering and paying for the test and the veterinarian (i.e., the clinical entity) is in charge of collecting the sample and delivering the results of the test. Thus, a prescription for ordering the medical test from the veterinarian (i.e., the clinical entity) is obviated, which lowers commercial and logistical barriers for the owner (i.e., the consumer entity) to order the test.

In one embodiment, the systems and methods described herein include: (i) allowing a pet owner (i.e., the consumer entity) to order a blood testing kit online and pay for a blood collection at the same time without the need for a prescription; and (ii) enable the pet owner to schedule a blood collection with a veterinarian (i.e., the clinical entity) through a preferred network of veterinarians. More specifically, the consumer is in charge of ordering the kit but is then able to get the blood collected and shipped back to the testing lab through the preferred network. The consumer is not limited to the preferred network and can choose his or her own veterinarian.

In one embodiment, the system and methods herein coordinates the interaction between the consumer and clinical entities. The system and methods herein can be further configured to communicate the status of the test to the consumer and the clinical entity. For example, the system and methods herein can: (i) notify the consumer and clinical entities of confirmation of an appointment with the veterinarian; (ii) notify the consumer and clinical entities that the sample has arrived at the testing entity (e.g., a medical testing lab handling biological samples from animals); (iii) notify the consumer and the veterinarian that the sample has been processed by the lab; (iv) notify the consumer and clinical entities that a test report has been sent to the clinical entity and a result is available; and (v) coordinate an appointment with the clinical entity to receive the results of the test.

### ABBREVIATIONS AND DEFINITIONS

To facilitate understanding of the invention, a number of terms and abbreviations as used herein are defined below as follows:

Pets: As used herein, the term “pets” refers to a companion animal kept for a human's company, entertainment, or as an act of compassion, such as taking on a stray dog. Additionally, pets, as used herein, can refer to an animal used by military or police to aid in law enforcement activity (e.g., detecting contraband substance); service animals that aid the visually impaired humans, agricultural animals; and animals used for sporting, such as race horses or dogs. A list of pets include: dogs, cats, horses, rabbits, ferrets, pigs, gerbils, hamsters, chinchillas, rats, guinea pigs, horses, parrots, passerines, fowls, turtles, alligators, crocodiles, lizards, snakes, fish, freshwater snails, saltwater snails, frogs, tarantulas, and hermit crabs. The list is not exhaustive as other non-human animals may be pets to humans.

Liquid biopsy: As used herein, the term “liquid biopsy” refers to the collection of a sample and the testing the sample, wherein the sample is non-solid biological tissue such as blood.

Circulating free deoxyribonucleic acid (DNA): As used herein, the term “cfDNA” refers to DNA fragments released to the blood plasma. cfDNA can include circulating tumor deoxyribonucleic acid (ctDNA).

Circulating tumor deoxyribonucleic acid (ctDNA): As used herein, the term “ctDNA” refers to a tumor-derived fragmented DNA in the bloodstream that is not associated with cells.

Process: As used herein, the term “process” is a set of instructions, which are unambiguous specifications, for performing calculation, data processing, automated reasoning, and other tasks.

Medical tests: As used herein, the term “medical tests” refer to diagnostic tests, screening tests, and monitoring tests. Diagnostic tests are procedures performed to confirm or determine the presence of disease in an animal organism, such as nuclear medicine techniques to determine the presence of lymphoma or blood sugar measurements to determine the presence of diabetes in the animal organism. Screening tests are procedures used to detect or predict the presence of disease in at risk individuals within a defined group within a population of animal organisms. Monitoring tests are procedures used to monitor the progress of or response to medical treatments. The procedures can include: a diagnostic kit for containing materials for performing the diagnostic tests; a screening kit for containing materials for performing screening tests; or a monitoring kit for containing materials for performing monitoring tests.

Medical Testing Kits: As used herein, the term “medical testing kits” refer to diagnostic kits, screening kits, and monitoring kits. Materials in the medical test kits can include one or more of the following: (1) items for collecting or receiving samples for the medical test; (2) items for storing the samples for the medical test; (3) items for transporting the samples for the medical test between other items within the medical test testing kit; and (4) items for analyzing the samples for the medical test. The samples for the medical test are biological material(s) deriving from the pets. The biological material(s) can include: blood, urine, saliva, fur, tissue, scrapings (e.g., plaque scraped from a tooth for dental diagnostic tests), and other fluid or solid materials from the pet. Test tubes; vials; syringes; needles which attach directly or indirectly to the body of the syringes; scrapers, and test strips with indicators (e.g., urinary test tract infection test strips) may be items for collecting or receiving the samples for the medical test (i.e., materials (1)). Storage containers can store or maintain the integrity of the samples for medical test for a later point in time (i.e., materials (2)). Plastic tubing (which connects needles with the body of the syringe) and paths for capillary action (which transports fluidic biological samples in testing strips) may be items that can transport the samples for the medical test (i.e., materials (3)). Indicators may be items which the sample for the medical test interact with to generate an analysis (i.e., materials (4)). Stated another way, analysis of the sample is performed for the medical test, which can indicate the presence or absence of the disease. The items listed above are not an exhaustive list and other items can be in the medical testing kit without departing from the scope of the invention. Depending on the medical test, the medical testing kit can incorporate any combinations of items among materials (1), materials (2), and materials (3), and materials (4). An example testing kit can incorporate items among materials (1), materials (2), and materials (3), and not incorporate items among materials (4). The items among materials (1)-(4) can be designed for specific functions, such as test tubes that can stabilize ctDNA for liquid biopsy applications.

### System Overview

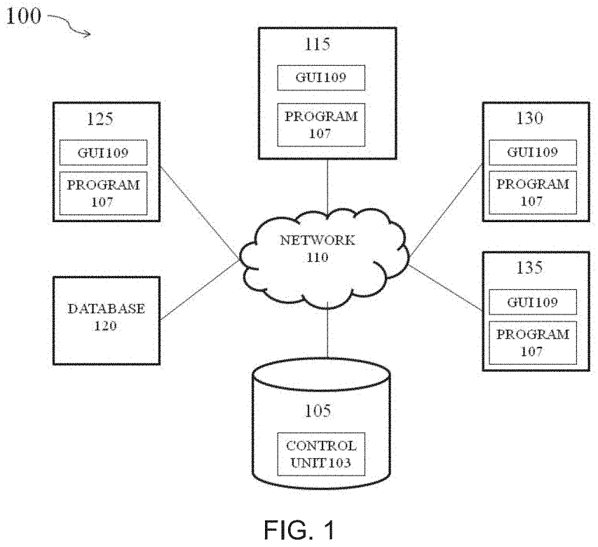

FIG. 1 shows an embodiment of an electronic system or environment 100 for managing and ordering medical tests. Components of computing environment 100 may communicate with one another over a network 110. For example, the network 110 connects to a server 105, a consumer device 115, a database 120, a clinical device 125, a logistics device 130, and a testing device 135. Some of these components attached to the network 110 are terminal nodes in which links are connected to allow telecommunication between the terminal nodes. For example, within the computing environment 100, the terminal nodes are server 105, consumer device 115, database 120, clinical device 125, logistics device 130, and testing device 135. Network 110 can be the Internet, a telephone network, a global Telex network, computer networks, and so forth. The structure of network 110 to route signals and the associated message include but are not limited to wide area networks (WAN), metropolitan area networks (MAN), local area networks (LAN), internet area network (IAN), campus area networks (CAN), and virtual private networks (VPN).

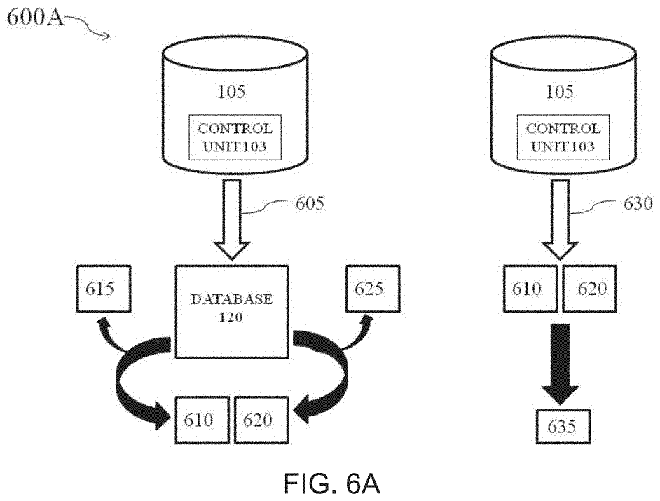

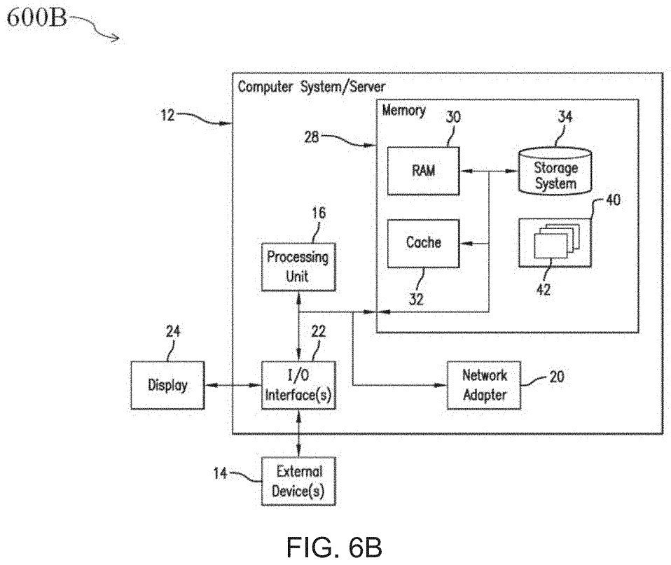

Consumer device 115, clinical device 125, testing device 130, and logistics device 135 may be electronic systems that include software, hardware and processors for carrying carry out various operations within the system. Each of these devices is described in more detail with respect to FIG. 6B. In one embodiment, these devices can be a laptop, desktop, tablet, smart phone, or any type of electronic system amenable to computer programming. Server 105 is a computer system that may include a control unit 103 for controlling the programs and commands within the server and could provide the direct-to-consumer distribution channel herein.

Database 120 is an organized collection of data stored and accessed by control unit 103 to form part of a database management system. The contents of database 120 may include: (i) clinical entities, logistic entities, and testing entities that have an agreement with the coordinating entity; (ii) clinical entities, logistic entities, and testing entities which do not have an agreement with the coordinating entities; and (iii) research findings pertaining to the care of pets. Database 120 can reside within control unit 103 or instead communicate from outside of control unit 103. The clinical entities which have an agreement with the coordinating entity are referred to as preferred providers.

Consumer device 115 may be operated by the consumer entity, such as a human individual owning and/or taking care of a pet. Clinical device 125 may be operated by the clinical entity, such as a veterinarian, pathologist, or professional equivalent trained to provide healthcare to a pet or other animals. Logistics device 135 may be operated by the logistics entity, such as a specimen handling company or a company delivering the medical testing kit to the consumer entities. Database 120 may be accessed by server 105. Testing device 135 may be operated by the testing entity, such as a medical testing laboratory.

Server 105 is operated by a coordinating entity that manages the server and supporting data. The clients supported by server 105 are: (i) database 120; and (ii) the operators of devices 115, 125, 130, and 135. Control unit 103 resides within server 105 and communicates with units of program 107 in consumer device 115, clinical device 125, testing device 130, and logistics device 135. Program 107 can be a mobile application or browser program in contact with server 105 via control unit 103. Control unit 103 processes instructions shared between the coordinating entity, consumer entity, logistics entity, and testing entity by using filtering processes, artificial intelligence (AI) techniques, and data analytics.

By the consumer entity ordering medical tests, the activities needed to assess the health of the pet of the consumer entity are organized by: (i) scheduling appointments with the clinical entity; (ii) pickups and deliveries by the logistics entity; and (iii) sending samples for the medical testing kit to a testing entity. After analyzing the results from the samples for the medical testing kits for assessing the health of the pet of the consumer, the consumer entity can be informed if the pet is at risk for cancer or other diseases. In this embodiment, control unit 103 sends contents from server 105 to program 107 in consumer device 115, clinical device 125, testing device 130, and logistics device 135. In turn, portals can be displayed in graphical user interface (GUI) 109 in consumer device 115 operated by the consumer entity, GUI 109 in clinical device 125 operated by the clinical entity, GUI 109 in testing device 130 operated by the testing entity, and GUI 109 in logistics device 135 operated by the logistics entity. Stated another way, there are displayed portals in the direct-to-consumer distribution channel herein that facilitate interaction between the different entities and streamline the activities needed to assess the pets for potential cancer in the early stages.

In one embodiment, there are filtering processes and machine learning/AI techniques applied to the data contents residing in database 120. The filtering processes and applied machine learning/AI techniques can distinguish between: (i) clinical entities, logistics entities, and testing entities that have an agreement with the coordinating entity; and (ii) clinical entities, logistics entities, and testing entities which do not have an agreement with the coordinating entities. The filtering process can aid in creating a list of clinical entities to be presented to the consumer entity. The AI and data analytics can be applied to present a further refined list of clinicians to program 107 in consumer device 115, which is in use by the consumer entity. The further refined list of clinicians (e.g., veterinarians, pathologists, and so forth) can be based on: (i) proximity of the clinical entity's office to the consumer entity; (ii) availability of a clinical entity in comparison to another clinical entity; and (iii) access to preferred testing entities, such as those with an agreement for the clinical entity. Other criterion can be used as well by the filtering process, AI techniques, and data analytics applied by control unit 103.

The AI techniques can include, for example, neural networks. Neural networks can be optimized for training with a very small number of parameters and do not need to make a priori assumptions on the properties of the underlying data. In the recent past, a variety of neural networks (both supervised and unsupervised) have been useful in detecting anomalies in medical images including Radial Basis Functions (RBF), Learning Vector Quantization (LVQ), Probabilistic Neural Networks (PNN), Hopfield networks, Support Vector Machines (SVM), Synchronized Oscillator Network, and Adaptive Resonance Theory (ART). More recently, Convolutional Neural Networks (CNNs) such as those described herein have become preferred models for coordinating the activities needed to assess the health of the pet of the consumer entity.

The neural network can include any combination of neural networks including, but not limited to, Perception Neural Network, Feed Forward Neural Network, Artificial Neuron, Deep Feed Forward Neural Network, Radial Basis Function Neural Network, Recurrent Neural Network, Long/Short Term Memory, Gated Recurrent Unit, Auto Encoder Neural Network, Variational AE Neural Network, Denoising AE Neural Network, Sparse AE Neural Network, Marakov Chain Neural Network, Modular Neural Network, Hopfield Network, Boltzmann Machine, Restricted BM Neural Network, Deep Belief Network, Deep Convolutional Network, Deconvolutional Network, Deep Convolutional Inverse Graphics Network, Generative Adversarial Network, Liquid State Machine Neural Network, Extreme Learning Machine Neural Network, Echo State Network, Deep Residual Network, Kohonen Self Organizing Neural Network, Support Vector Machine Neural Network, Neural Turing Machine Neural Network, Convolutional Neural Networks such as LeNet, AlexNet, ZF Net, GoogLeNet, VGG Net, Microsoft ResNet, and Region Based CNNs including, but not limited to, Fast R-CNN, Faster R-CNN, R-FCN, Multibox, SSD, and YOLO, amongst others which are known to those of skill in the art.

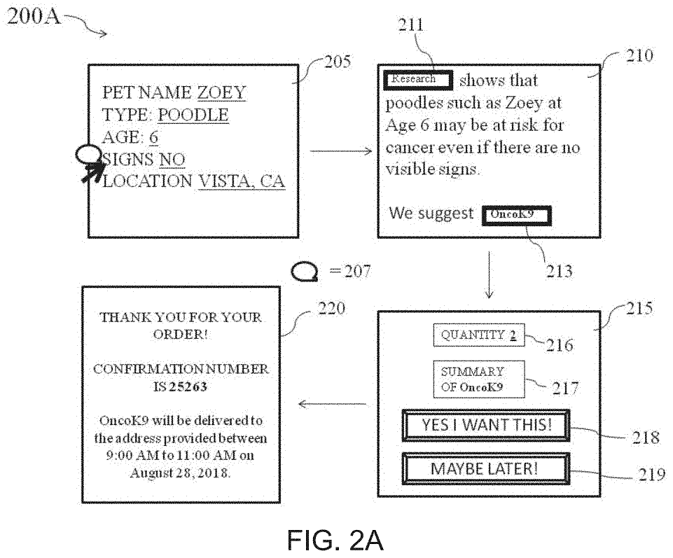

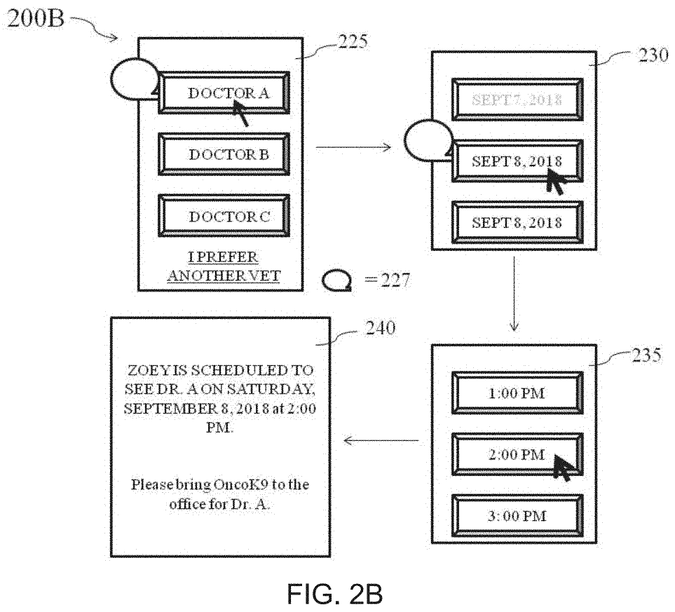

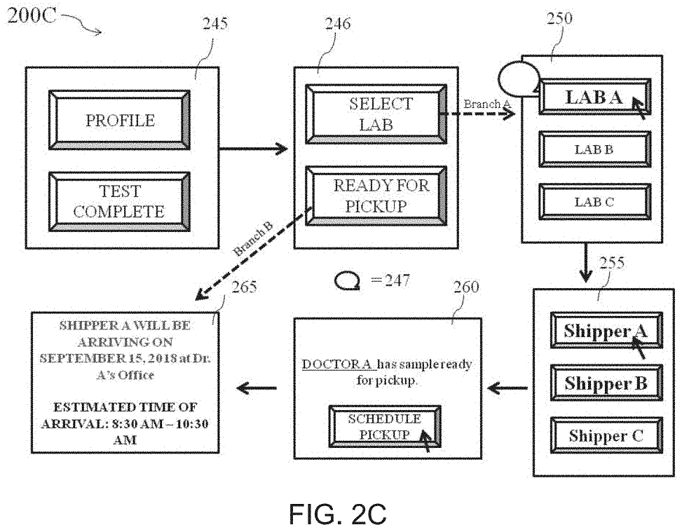

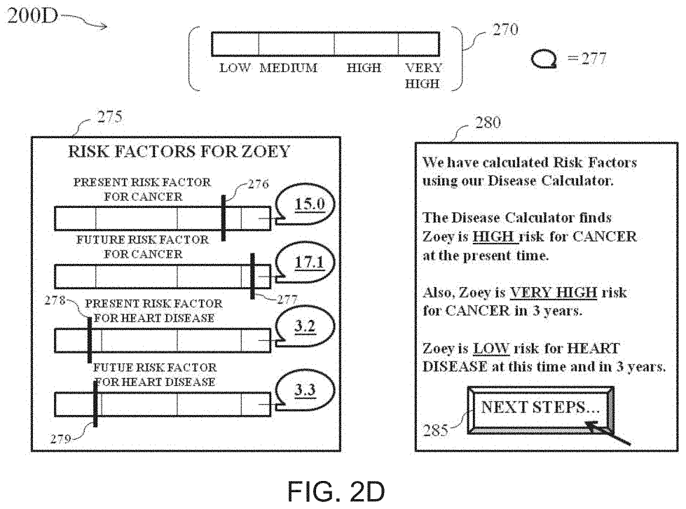

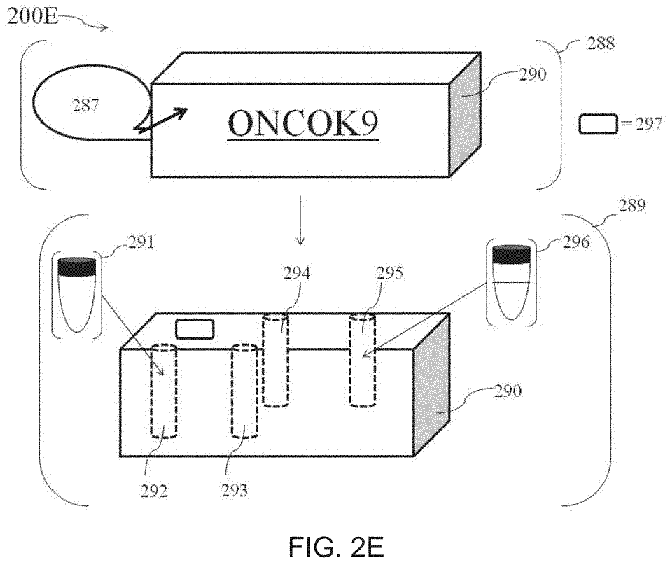

FIG. 2A, FIG. 2B, FIG. 2C, FIG. 2D, and FIG. 2E are interface environments that can be generated and used to display portals in the direct-to-consumer distribution channel herein. All dates shown in the figures and described below are shown as examples and should not be considered as limiting.

Upon control unit 103 determining that a consumer entity has ordered a kit for the medical test (e.g., a diagnostic kit, a screening kit, and a monitoring kit), control unit 103 instructs program 107 in device 135 operated by the logistics entity to ship and deliver the kit for the medical test to the consumer entity.

Referring now to FIG. 2A, a consumer entity is using program 107 via a user interface. The consumer entity may enter data regarding their pet into the user interface to help in deciding whether to order a medical testing kit for their pet. The process 200A is shown as moving through several interface screens to obtain the data from the consumer entity. For example, interface 205 may be presented in GUI 109 in consumer device 115. The consumer entity enters the following information in the categories interface 205: the name (of pet) as ZOEY, type (of pet) as POODLE, the age (of the pet) as 3, signs (of the pets having symptoms indicative of cancer or disease) as NO, and the location (of the pet and the consumer entity) as VISTA, Calif. An information bubble 207 may be generated by control unit 103 when the cursor in consumer device 115 is placed over one of the categories. For example, the cursor (which is the bolded arrow) is placed on the signs category in interface 205. Information bubble 207 contains a more detailed description of what the signs category is referring to. Information box 207 lists certain symptoms to look for, such as excessive panting by a dog or if the dog has been atypically lethargic for a two-week time period. Program 107 in device 115 sends the entries in interface 205 to control unit 103. In turn, the filtering process and AI techniques can be applied by control unit 103 on database 120, which contains research findings.

After entering information into interface 205, the consumer entity is presented with interface 210, which may be based on the combination of the entries in interface 205 and data from research findings extracted from database 120. Interface 210 displays to the consumer entity that “Research shows that poodles such as Zoey at Age 6 may be a risk for cancer even if there are no visible signs”. Stated another way, the entries for the categories in interface 205 are incorporated into interface 210. Control unit 103 determines a likelihood of disease for the pet, based on the research finding and entries for the categories in interface 205. By applying the filtering process and AI techniques, control unit 103 determines which diseases the pet is at potential risk of having cancer at the present time and at future times. For example, a first journal reports that dogs at age 6 have higher confirmed cases of cancer than cats at age 6. In the same example, a second journal reports that cats at age 6 have higher confirmed cases of heart disease than dogs at age 6. In turn, control unit 103 applies the filtering process and AI techniques on the first journal, the second journal, and the entries for the categories in interface 205 to make the determination that the poodle Zoey, which is a dog as opposed to a cat, is more likely at risk for cancer than heart disease. Based on the entries for the categories in interface 205 and analysis of findings in research journals, a risk factor can be determined, as described in more detail with respect to FIG. 2D.

In GUI 109 in device 115, the consumer entity can access buttons 211 and 213, as shown in the interface 210. By pressing button 211, the link to the research finding is opened and discloses the suggested medical test, which is presented in button 213. By pressing button 211, the link to a description of OncoK9™ found in server 105 is opened. The consumer entity reads the description of OncoK9™ and deems the medical test as beneficial to his or her pet. Control unit 103 generates interface 215 in response to receiving the order from the consumer entity to purchase a medical test contained within the medical testing kit. In this instance, the pet is a dog and the medical test suggested is OncoK9™. In another instance, the pet is a cat and the medical test suggested is OncoFeline™

Following the selections made on interface 210, interface 215 is displayed on consumer device 115. In GUI 109 of consumer device 115, the consumer entity indicates two units of the medical testing kit are going to be ordered in category 216. In category 217, button 217 has a link to a summary of the OncoK9™ medical testing kit. Upon pressing button 217, consumer entity is presented with properties of OncoK9™ in GUI 109 of consumer device 115. Example of properties comprise: OncoK9™ is a liquid biopsy test for dogs; the price of the medical testing kit; the sample type is blood; the technology platform is next-generation sequencing; the medical test contained within the medical testing kit is directed for early detection and targeted treatments; and the medical test contained within the medical testing kit can detect 90% cancers. In contrast to OncoK9™, other medical tests collect tissue or urine instead of blood. Also, the other medical tests cannot detect as many cancers as OncoK9™. Blood would be easier to obtain from a dog than tissue or urine. Accordingly, control unit 103 can suggest a more convenient medical test for the consumer entity to assess the health of the dog.

The consumer entity can press button 218 or button 219. If button 219 is selected and pressed, then control unit 103 leaves interface 215 and returns back to interface 205. If button 218 is selected and pressed, then the consumer entity is taken to a payment page (not depicted). In response to control unit 103 processing payment as being received from the consumer entity, control unit 103 sends a confirmation message shown in interface 220, as depicted in GUI 109 of consumer device 115. Additionally, in response to control unit 103 receiving payment, control unit 103 finds a shipper among a list of logistics entities to deliver the two medical testing kits containing OncoK9™ to the consumer entity by Aug. 28, 2018 between 9:00 AM and 11:00 AM.

For the purposes of clarity, a cursor is drawn as a thick bold arrow, as used in personal computers, to select buttons in a GUI. For smart phones and tablets, a cursor may not be present in a GUI. However, the user of the smart phones or tablets can use haptic feedback mechanisms with the GUI for selecting the buttons in the GUI.

Referring now to FIG. 2B, interface 225 is shown displaying a list of clinician entities that have an arrangement with the coordinating entity or are selected to be the clinical entities to be shown to the consumer for the area where consumer entity resides. In interface 225, doctors A-C are listed as clinical entities in button form for which the consumer entity can schedule an appointment for collecting the blood sample for the medical testing kit. Information bubble 227 appears when the cursor is placed over a button. The cursor, which is a thick bold arrow, is placed over Doctor A and information bubble 227 appears in GUI 109 of device 115. The consumer entity can read the contents of information bubble 227, such as location of Doctor A. However, the consumer entity does not have to select the list of doctors, as provided by the filtering process applied by control unit 103 (doctors A-C in FIG. 2B). The consumer entity may prefer a clinical entity which does not have an arrangement with the coordinating entity. If the consumer entity prefers a clinical entity besides the clinical entities in interface 225, the consumer entity may select the link, which is underlined, “I prefer another vet”. The consumer entity is taken to a GUI operated by control unit 103. In this GUI, control unit 103 prompts the consumer entity to enter in the information of the clinical entity that he or she prefers to take the pet for collecting the sample.

Upon pressing the button for Doctor A, control unit 103 displays an interface 230. The buttons in interface 230 have a list of dates for going to the selected doctor, which is Doctor A in this instance. Doctor A is unavailable on Sep. 7, 2018 and thus the button for Doctor A is grayed out. Grayed out buttons cannot be selected. The buttons for Sep. 8, 2018 and Sep. 9, 2018 are not grayed out so they can be selected by the consumer entity. The cursor, which is a thick bold arrow, can be placed over the buttons for Sep. 8, 2018 and Sep. 9, 2018 and information bubble 227 appears in GUI 109 of device 115. In interface 230, the cursor is placed over the button for Sep. 8, 2018 and information bubble 227 has a list of times the consumer entity can come to the office of the clinical entity.

When the consumer entity clicks on the button for Sep. 8, 2018, control unit 103 generates interface 235. Buttons of available times are presented to GUI 109 of device 115. The available times are not grayed out buttons. In interface 235, the buttons for 1:00 PM, 2:00 PM, and 3:00 PM are not grayed out and thus either one of these times can be selected. The consumer entity places the cursor, which is a thick bold arrow, on the button for 2:00 PM. Upon pressing the button for 2:00 PM, control unit 103 generates interface 240 which indicates that the pet's name from the categories in interface 205 is scheduled to see the doctor selected in interface 225 on the day selected in interface 230 at a time selected in interface 235. Additionally, the medical test selected from interface 210 and 215 is indicated in interface 240.

As shown in FIG. 2C, the control unit 103 generates interface 245 which is sent to program 107 in device 125, which is in use by the clinical entity. In this instance, Doctor A is the selected clinical entity from interface 225. A profile for the pet, as entered by the consumer entity, can be retrieved by pressing the button for the profile in interface 225. If control unit 103 validates the profile, the button for the profile is not grayed out. In an embodiment, once the test request form is uploaded by control unit 103 and the blood sample is collected from the pet for the medical tests, the button for test complete is not grayed out. The clinical entity can press the button for test complete to initiate interface 246. In interface 246, clinical entity can decide if he or she wants to: (Branch A) select a lab for shipping the sample by pressing the “select lab” button; or (Branch B) bypass selecting a testing entity and logistics entity by pressing “ready for pickup” button.

If the “select lab” button is selected, control unit 103 generates interface 250 in GUI 109 in device 130 which contains a list of testing entities or labs A-C for which the sample can be sent to. The testing entities in interface 250 have an agreement with the coordinating entity, based on accuracy and reliability of testing. When the cursor, which is a thick bolded arrow, is placed on the button for LAB information box 247 appears and LAB A is bolded. Information box 247 contains the address information and schedule availabilities for each lab in the listed buttons. Upon the clinician entity pressing the button for LAB A, control unit 103 generates interface 255, which is a list of logistic entities that can transport the sample from the selected clinical entity to the selected testing entity. The coordinating entity or control unit 103 applies the filtering process and/or AI techniques to select a logistic entity among Shipper A, Shipper B, and Shipper C. In response to selecting the button for Shipper A, control unit 103 generates interface 260, which indicates Doctor A from interface 225 has been selected and has a button to SCHEDULE PICKUP in GUI 109 in device 125. Control unit 103 determines a pickup time and date, based on the sample for the medical testing kit. Some medical tests or medical testing kits are more prone to degradation than others, so control unit 103 connects to program 107 in device 135 operated by the logistics entity, which can promptly collect the sample. In turn, control unit 103 generates a notification that the selected logistics entity, such as Shipper A in interface 265, will be arriving the day after collecting the sample at the selected clinical entity and the estimated time of arrival. The notification in interface 265 is sent to the selected clinical entity, testing entity, and logistics entity.

If the “ready for pickup” button is selected, control unit 103 generates interface 265. Control unit 103 applies filtering processes and AI techniques to find: (i) a testing entity to receive the shipped sample; (ii) the logistic entity that picks up the sample from the clinical entity and ships the sample to testing entity; and (iii) estimated time of arrival of the logistics entity at the clinical entity for pickup. As stated above, control unit 103 can use the filtering processes to make the determination pertaining but not limited to the following: (D1) the stability of the medical test and medical testing kit; (D2) distance of the testing entity from the clinical entity; (D3) the capability of the logistics entity (e.g., availability and reliability of Shipper A, B, and C); and (D4) other factors which can impact the pickup or delivery of the sample (e.g., adverse weather conditions and other events which can inhibit travel). In embodiment, control unit 103, based on D1-D4, determines that Shipper A is best suited logistic entity to pickup the sample from the office of the clinical entity (which is, for example, Doctor A) to the laboratory of the testing entity (which is, for example, LAB A). Interface 265 can be generated by control unit 103, which states the time the logistic entity arrives at the clinical entity for picking up the sample. In this situation, a prelabeled container with the sample can be placed in a pickup at the office of clinical entity for shipment to the testing entity.

Based on the filtering process and AI techniques applied on the entries in the categories of interface 205 and research findings, a disease calculator operated by control unit 103 determines a risk factor for a disease within a timeframe. The risk factor can be a numeric value, where a range of numeric values can be associated with, for example, low risk (risk factor of 0-4), medium risk (risk factor of 4.1-11.0), high risk (risk factor of 11.1-15.5), or very high risk (risk factor of 15.6 to 20) for the disease within the timeframe. The timeframe can be the present time or a future period of time. The future period of time can be configured by the coordinating entity or extracted from research journals in response to control unit 103 applying filtering processes and AI techniques on the research journals. The ranges and associated risk levels are depicted as scale 270.

After determining the risk factor, control unit 103 generates interface 275 in FIG. 2D. Interface 275 is sent to program 107 in device 115. Information boxes 277 reside next to scale 270 for each disease and accompanying timeframe. For example, risk factors are determined to be: 15.0 for cancer at the present time, which is associated with high risk; 17.1 for cancer at a future time, which is associated with very high risk; 3.2 for heart disease at the present time, which is associated with low risk; and 3.3 for heart disease at a future time, which is associated with low risk. Bars 276, 277, 278, and 278 are depicted in interface 275 to visualize the relative risk factors for the different diseases and timeframes. Based on the shift of the location of bar 276 in the high risk area to bar 277 in the very high risk area, control unit 103 has determined that the pet appears to be at increasing risk of cancer and that immediate action should be taken. Based on the shift of the location of bar 278 to bar 279, control unit 103 has determined that the pet appears to be at low risk for heart disease in the present time and the future time. The shift is more pronounced for the cancer in comparison to heart disease. Control unit 103 also determines that the pet is at greater risk for cancer than heart disease. In information boxes 277, the risk factors are underlined and represent links that can be clicked on.

After clicking on any of the links in information boxes 277, control unit 103 generates interface 280, which is sent to program 107 in device 115. In GUI 109 in device 115, control unit 103 can indicate that the pet, Zoey, is: (i) at high risk for cancer at the present time; and (ii) at very high risk for cancer at the future time of 3 years. Additionally, control unit 103 can indicate that Zoey is at low risk for heart disease at present and future times. Button 285 is a link to the recommended course of action for the consumer entity. After clicking on button 285, control unit 103 sends interface 210 in FIG. 2A and/or interface 288 in FIG. 2E to program 107 in device 115.

As shown in graphical display 200E, a virtual box labeled with OncoK9™ is displayed in consumer device 115 as an assembled virtual medical testing kit 290. Information box 287 appears when a cursor, as depicted as a thick bold line, is placed on medical testing kit 290. Information box 287 provides more information on OncoK9™ wherein the information is identical to the information provided by clicking on button 217 in FIG. 2A. The underlined text OncoK9™ in virtual medical testing kit 290 is a link from interface 288 to interface 289. In interface 289, virtual medical testing kit 290 is disassembled and graphical representations of the contents of medical testing kit 290 are able be taken out of the box. The contents and medical testing kit 290 can be viewed from different angles and perspectives using control unit 103.

Virtual medical testing kit 290, which is a graphical representation of an actual medical testing kit, can incorporate items among materials (1), materials (2), and materials (3), and materials (4), as described above. Box 297 contains a bar-code generated by control unit 103. The filtering process is applied by control unit 103 on the selected medical test, ordered medical kit, and/or selected clinical entity to generate the bar code. The bar code, as generated by control unit 103, is a collection of parallel lines and accompanying numeric code customized for each profile and ordered medical kit. Thus, the bar-code can further tele-communicatively connect units of program 107 in consumer device 115, clinical device 125, testing device 130, and logistics device 135 with control unit 103 in server 105 to each other. The bar-code is amenable to laser scanning or other types of optical scanners applied on the collection of parallel lines to retrieve the contents of a profile (as described above) by the coordinating, clinical, testing, and logistics entities. The accompanying number may optionally be entered in manually in program 107 in clinical device 125, testing device 130, or logistics device 135 to retrieve the contents of the profile.

Prior to collecting the blood sample, test tube 291 (i.e., a test tube body and connected cap) can be stored in chamber 292. Test tube 291 can make a supple fit into chamber 292. By making a supple fit, this guards test tube 291 against physical damage. Covered needles; syringes; and tubing (which can operatively connect test tube 291, needles, and syringes to each other) can be optionally stored in chambers 293 and 294. In response to collecting the blood sample into test tube 291, test tube 291 resembles test tube 296. Test tube 296 can make a supple fit into chamber 295. By making a supple fit, this reduces the likelihood of disturbing the blood sample and guards test tube 296 against physical damage. Additionally, chamber 295 can maintain a colder temperature and resist heat to prevent thermal degradation of the sample. Stated another way, the sample in test tube 296 maintains the properties needed to ensure an accurate test upon reaching the testing entity. After collecting the sample, the clinical entity may re-scan the bar-code or manually re-enter the accompanying number to confirm that a sample has been collected. This confirmation may be a trigger used by control unit 103 in server 105 to initiate interface 255 in FIG. 2C. The logistics entity may perform multiple iterations of scanning and re-scanning.

Control unit 103 may analyze the patterns of scanning and re-scanning using the filtering process and AI techniques to ensure: (i) the medical testing kit, upon delivery to the consumer entity has not been tampered; and (ii) the sample, upon delivery to the testing entity, has not been tampered. Thus, the bar-code in combination with the activities of the coordinating, consumer, clinical, testing, and logistics entities, as processed by control unit 103, maintains the integrity of the medical test in the direct-to-consumer distribution channel herein.

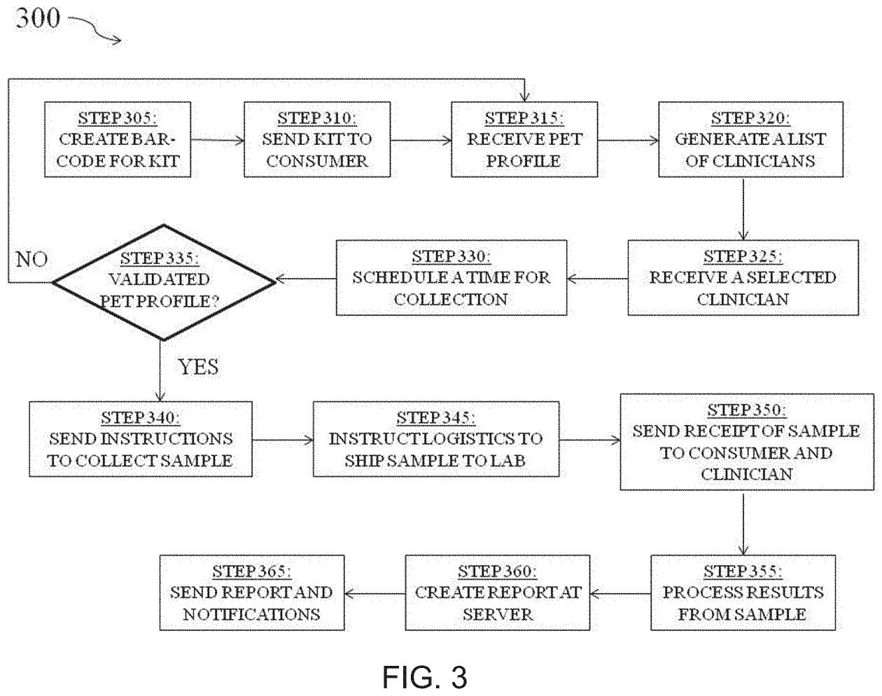

Referring now to FIG. 3, control unit 103 in server 105 executes the operational steps in flowchart 300. To initiate the operation steps in flowchart 300, a consumer entity orders a medical testing kit for his or her pet using the portal created by connecting control unit 103 and program 107 in device 115.

In step 305, control unit 103 creates a bar code for the medical testing kit. The medical testing kit may also include but is not limited to: the medical test; one or more tubes for storing blood samples; vials; instructions for filling out to the pet profile online; instructions for collecting the blood sample; a set of covered needles; alcohol wipes; and a return-shipping container. The bar code may be attached to one or more components of the medical testing kit.

In step 310, control unit 103 sends the medical testing kit to the consumer (entity) by instructing the logistics entity to ship the bar-coded medical testing kit to the consumer entity (as depicted in FIG. 2E), based on the selected medical testing kit from FIG. 2A. This is completed within a time frame of 2-3 days.

In step 315, control unit 103 receives a pet profile of the pet taken care of by the consumer entity. Pet profile information comprises: weight, age, name, breed, medical history, and other information pertaining to the pet. The medical testing kit which has been sent to the consumer entity may need to be promptly used or else the efficacy of the medical testing kit decreases. This information can be included in the pet profile. For instances where the medical test is OncoK9™ or OncoFeline™, the shelf life for the medical testing kit for implementing the medical test is 6 months. By control unit 103 in server 105 applying the filtering process and AI techniques, this information is accounted for. More specifically, a time frame of 6 months can be used to schedule and coordinate activities between the clinical, logistic, and testing entities.

In step 320, control unit 103 generates a list of clinicians (i.e., clinical entities that are preferred by the coordinating entity that runs server 105). In step 325, control unit 103 receives a selected clinician (i.e., the pressed button for a clinical entity in interface 225). This is the clinical entity that the consumer entity is going to take his or her pet to the office of the clinical entity for collecting a sample needed for the medical test.

In step 330, control unit 103 schedules a time for collection of the sample for the medical testing kit, as depicted by example in interfaces 230 and 235. While the scheduling is performed entirely online in this instance, control unit 103 in server 105 can connect to a link for a phone number to a clinical entity. The consumer entity can dial the phone number to schedule an appointment.

At the office of the clinical entity at step 335, control unit 103 determines if the pet profile is validated, based on an assessment done by the clinical entity. The first iteration is performed by the clinical entity and used to train the AI techniques applied by control unit 103. The AI techniques may also be applied on the pet profile to identify any inconsistencies or other red flags indicative of false or misleading information. If control unit 103 identifies inconsistencies or other red flags, control unit 103 does not validate the pet profile (i.e., the NO branch from step 335). In turn, control unit 103 reverts to step 315 to receive a new pet profile. For example, the clinical entity may edit the fields of the pet profile during the first iteration. After subsequent iterations, the trained AI techniques can identify errors or inconsistencies entered by the clinical or consumer entity. If control unit 103 or clinical entity validate a pet profile (i.e., the YES branch from step 335), control unit 103 sends instructions on how to collect a sample for the medical testing kit in step 340. These steps of validating the pet profile with the aid of control unit 103 in server 305 may take place over a period of 15-30 minutes.

The instructions may indicate for the OncoK9™ medical testing kit one or more of the following: 1) how much blood should be drawn for each tube; and 2) how to send sample(s) to the testing entity within 24 hours of collecting the sample(s). After receiving these instructions from control unit 103, the clinical entity collects the sample.

After collecting the sample, such as blood for the medical test, the clinical entity inform that the sample has been collected (i.e., pressing the button for test complete in interface 245). This notifies control unit 103 that a logistics entity is needed to ship the sample from the clinical entity to the testing entity (i.e., interfaces 255, 260, and 265). The testing entity is preferably a central laboratory connected to control unit 103 residing in server 105. In step 350, control unit 103 sends confirmation of sample receipt to the consumer entity and clinical entity.

In step 355, control unit 103 processes the results from the sample. Mutations, circulating tumor DNA fragments, and other parameters, as instructed by control unit 103, are determined and assessed by control unit 103. The filtering process, AI techniques, and data analytics are applied on the pet profile and the parameters determined from the samples to assess the health of the pet. Control unit 103 highlights values for parameters that may be indicative of the pet having cancer. At step 360, an electronic report in, for example PDF format, is created in server 105 by control unit 103, wherein the electronic report is in a secured and encrypted format. Based on configured settings, control unit 103 notifies the clinical entity of the electronic report availability in step 365. Control unit 103 sends the results in the electronic report to clinical entity. The clinical entity may modify the results and deliver the results to the consumer entity. Control 103 could also send the results in the electronic report directly to the consumer entity skipping the clinical entity. Thus, an assessment of the heath of the pet, based on the sample in the medical testing kit, can be made.

By performing these operational steps of flowchart 300, control unit 103 in server 105 provides knowledge on the pet's health state in a convenient and streamlined fashion. New customers can be acquired by the clinical entity without the inefficiencies, which may include: (i) incurring acquisition costs; (ii) collecting or maintaining wasteful inventory; and/or (iii) completing time-consuming paperwork. Furthermore, control unit 103 can create the direct-to-consumer distribution channel herein, in which the consumer entity does not have to go to the clinical entity for a prescription for a medical test, which often requires a prescription. The systems and methods herein allow the clinical entity to acquire the ownership of the clinical-patient relationship with multiple-touch points. The sample collection support process in the systems and methods herein drives customer adoption and retention of the medical test suggested by control unit 103.

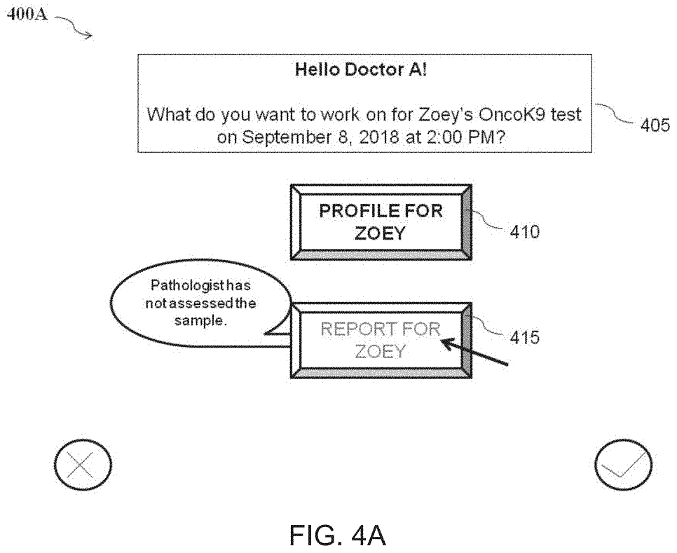

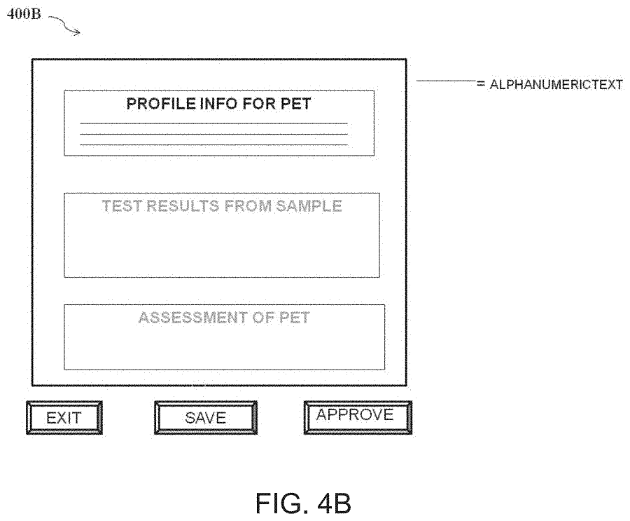

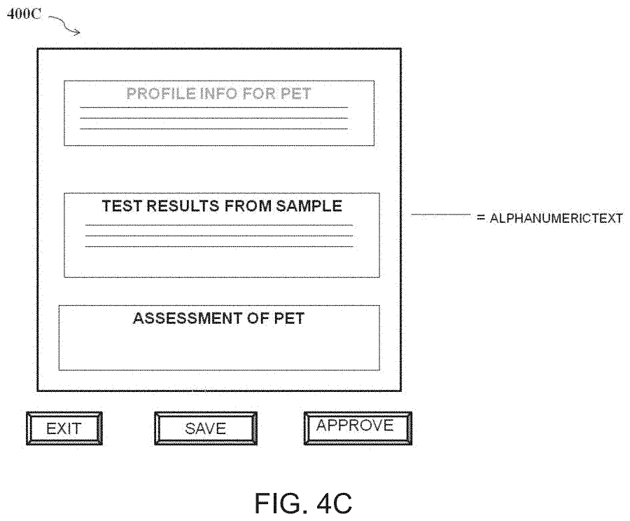

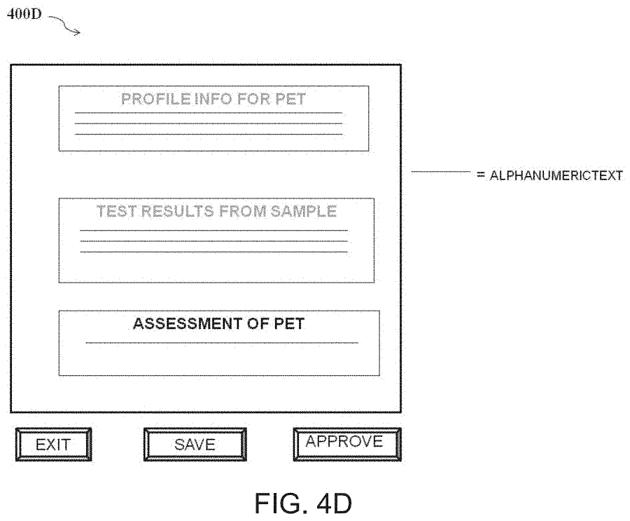

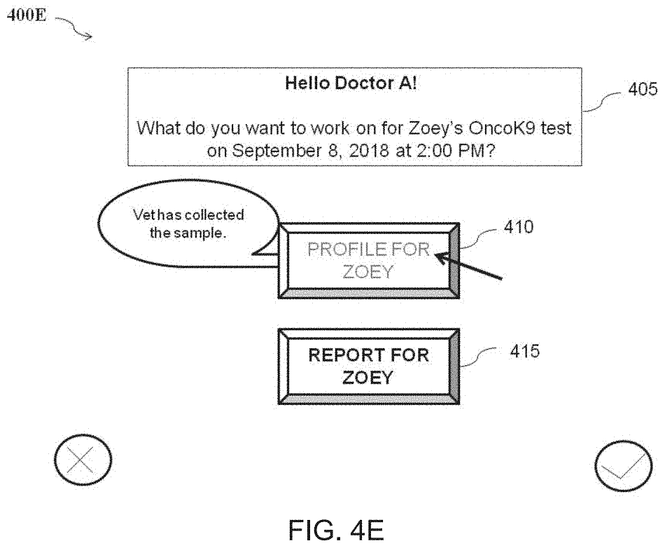

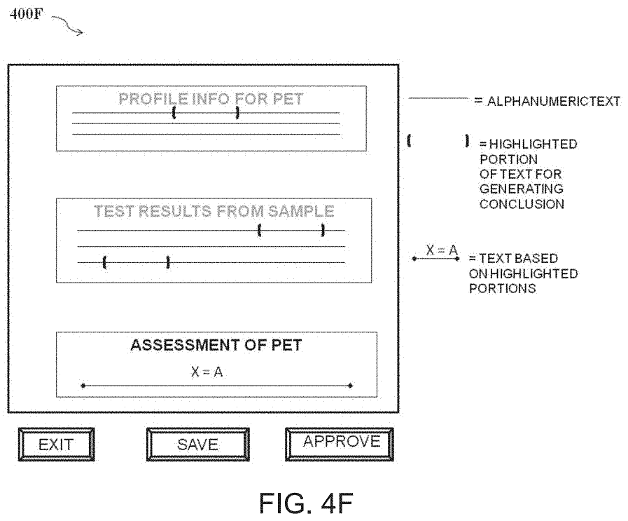

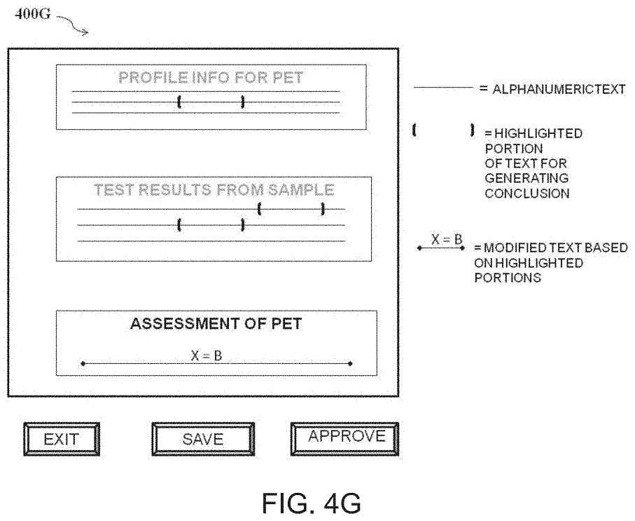

Referring now to FIG. 4A, an interface 400A appears in program 107 of clinical device 125 operated by a veterinarian (i.e., the clinical entity). In an embodiment, Doctor A is the selected veterinarian by consumer entity and the date the consumer entity brings his or her pet, Zoey, to the clinical entity for obtaining the sample for the selected medical test, OncoK9™. As depicted in interface 400A, the selected veterinarian on the date and time is greeted with a message 405. A button 410 in bolded and thus can be selected by the veterinarian, whereas a button 415 is grayed and thus cannot be selected the veterinarian. This is a mechanism that may be implemented by control unit 103 to ensure that proper protocols are continuously practiced by the disparate entities. The consumer entity at this point has brought his or her pet to the clinical entity and thus a report assessing the health of the pet is not available. The cursor can be placed over button 410 or 415. When the cursor is placed over button 415, as depicted in interface 400A, an information box appears which states “Pathologist has not assessed the sample.” If the veterinarian wants to exit program 107, the veterinarian clicks on the “x” in the bottom left hand corner of interface 400A. If the veterinarian wants to proceed to interface 245 (as described above), the veterinarian clicks on the “✓” in the bottom right hand corner of interface 400A. The veterinarian can modify the profile of the pet, Zoey, by clicking on button 410.

In response to clicking on button 410, control unit 103 sends interface 400B, as depicted in FIG. 4B, to the veterinarian using program 107 in clinical device 125. In an embodiment, there may be three sections in interface 400B: (i) the profile info for the pet; (ii) test results from the sample; and (iii) assessment of the pet. The profile info for the pet has been previously entered by the consumer entity and thus has alphanumeric text as contents in this section. The profile info for the pet is bolded, whereas the test results from sample and assessment of pet are grayed out and are absent of alphanumeric text. The grayed out sections are viewable and not modifiable by the clinical entity, whereas the profile for the pet is bolded and modifiable by the veterinarian obtaining the sample from the pet. This is another aspect of the mechanism that may be implemented by control unit 103 to ensure that proper protocols are continuously practiced by the disparate entities. More specifically, the test results from the sample and assessment of pet cannot be modified at this point because the sample has not been sent to the testing entity and are absent of contents. Additionally, there are buttons on the bottom of interface 400B, which can be pressed to: (i) close out interface 400B via an exit button; (ii) save modifications to the profile info for the pet via a save button; and (iii) proceed to interface 245 via an approve button.

Referring now to FIG. 4C, the sample has been sent to the selected testing entity. In an embodiment, control unit 103 sends interface 400C, as depicted in FIG. 4C, to the testing entity using program 107 in testing device 130. In an embodiment, there may be three sections in interface 400C: (i) the profile info for the pet; (ii) test results from the sample; and (iii) assessment of the pet. The grayed out sections are viewable and not modifiable by the testing entity, whereas the bolded section is viewable and modifiable by the testing entity. The profile info for the pet has been previously entered by the consumer entity and thus has alphanumeric text as contents in this section. The profile info for the pet is grayed out to a pathologist and other staff members of the testing entity, whereas the test results from sample are bolded and the assessment of the pet are bolding. The testing entity, which has received the sample and performed the tests on the sample, can enter in the results as alphanumeric text in the test result from sample section. By virtue of being grayed out, the contents of the profile information for the pet may be viewable but not modifiable. This is yet another aspect of the mechanism that may be implemented by control unit 103 to ensure that proper protocols are continuously practiced by the disparate entities. Further, the profile info of the pet cannot be tampered, which bolsters the integrity of the assessment of the pet that needs to determine. Additionally, there are buttons on the bottom of interface 400C, which can be pressed to: (i) close out interface 400C via an exit button; (ii) save modifications to the test results from the sample via a save button; and (iii) proceed to interface 400D via an approve button.

Referring to FIG. 4D, the sample has been analyzed and the results are assessed by the pathologist at the testing entity. In an embodiment, control unit 103 sends interface 400D, as depicted in FIG. 4D, to the testing entity using program 107 in testing device 130. In an embodiment, there may be three sections in interface 400D: (i) the profile info for the pet; (ii) test results from the sample; and (iii) assessment of the pet. The profile info for the pet has been previously entered by the consumer entity and thus has alphanumeric text as contents in this section. The profile info for the pet and test results from sample are grayed out to a pathologist and other staff members of the testing entity, whereas the assessment of the pet is bolded. The grayed out sections are viewable and not modifiable by the testing entity, whereas the bolded section is viewable and modifiable. The testing entity, which has received the sample and performed the tests on the sample, can enter contents in the assessment of the pet section. Stated another way, the pathologist can enter his or her medical determinations in the assessment of the pet section. By virtue of being grayed out, the contents of the profile info for the pet and the test results from sample sections cannot be modified. This is yet another aspect of the mechanism that may be implemented by control unit 103 to ensure that proper protocols are continuously practiced by the disparate entities. The profile info and testing results cannot be modified and this further bolsters the integrity of the assessment of the pet. Additionally, there are buttons on the bottom of interface 400D, which can be pressed to: (i) close out interface 400D via an exit button; (ii) save modifications to interface 400D via a save button; and (iii) proceed to interface 400E via an approve button.

Referring to FIG. 4E, an interface 400E appears in program 107 of clinical device 125 operated by a veterinarian (i.e., the clinical entity). In an embodiment, Doctor A is the selected veterinarian by consumer entity and the date the consumer entity brings his or her pet, Zoey, to the clinical entity for obtaining the sample for the selected medical test, Onco9™. As depicted in interface 400E, the selected veterinarian on the date and time is greeted with a message 405. A button 415 in bolded and thus can be selected by the veterinarian, whereas a button 410 is grayed and thus cannot be selected by the veterinarian. This is a mechanism that may be implemented by control unit 103 to ensure that proper protocols are continuously practiced by the disparate entities. The testing entity at this point has received and analyzed the sample and thus a report assessing the health of the pet is available for the veterinarian. The cursor can be placed over button 410 or 415. When the cursor is placed over button 410, as depicted in interface 400E, an information box appears which states “Vet has collected the sample.” If the veterinarian wants to exit program 107, the veterinarian clicks on the “x” in the bottom left hand corner of interface 400E. If the veterinarian wants to proceed to interface 400F, the veterinarian clicks on the “✓” in the bottom right hand corner of interface 400E. The veterinarian can modify the report for Zoey, by clicking on button 415.

Referring to FIG. 4F, an interface 400F appears in program 107 of clinical device 125 operated by a veterinarian (i.e., the clinical entity). In an embodiment, control unit 103 sends interface 400F, as depicted in FIG. 4F, to the clinical entity using program 107 in clinical device 125. In an embodiment, there may be three sections in interface 400F: (i) the profile info for the pet; (ii) test results from the sample; and (iii) assessment of the pet. The profile info for the pet, test results from the sample, and assessment of the pet has been previously entered and thus has alphanumeric text as contents in these sections. The profile info for the pet and test results from sample are grayed out to the veterinarian, whereas the assessment of the pet is bolded. The grayed out sections are viewable and not modifiable by the testing entity, whereas the bolded section is viewable and modifiable. The clinical entity can enter contents and modify the assessment of the pet section. By virtue of being grayed out, the contents of the profile info for the pet and the test results from sample sections cannot be modified. This is yet another aspect of the mechanism that may be implemented by control unit 103 to ensure that proper protocols are continuously practiced by the disparate entities. Control unit 103 allows the veterinarian to apply highlighting brackets on the contents of the profile info for the pet and test results from the sample to modify the assessment of the pet. The assessment of the pet (X) is A, which based on criteria that impact the health of the pet, such as the age of the pet, lipid profiles, and so forth. The criteria may include: the contents and information in upper middle highlighting bracket in the profile info for the pet section; the upper right highlighting bracket in the test results from the sample section; and the lower left highlighting bracket in the test results from the sample section. Additionally, there are buttons on the bottom of interface 400F, which can be pressed to: (i) close out interface 400F via an exit button; (ii) save modifications to interface 400F via a save button; and (iii) proceed to publish the reports with the sections mentioned above via an approve button. The brackets which appear in interface 400G are absent in the published report, which can be in PDF form.

Referring to FIG. 4G, the veterinarian (i.e., the clinical entity) may think other criteria are more relevant in assessing the health of the pet. In an embodiment, control unit 103 sends interface 400F, as depicted in FIG. 4F, to the clinical entity using program 107 in clinical device 125. As stated above, the profile info for the pet and test results from the sample are grayed out and thus not modifiable. The upper middle highlighting bracket in the profile info for the pet section from interface 400F may shift to the lower middle highlighting bracket in the profile for the pet section in interface 400G. The lower left highlighting bracket in the test results from the sample section from interface 400F may shift to the center middle highlighting bracket in the test results from the sample in interface 400G. The lower left highlighting bracket in the test results from the sample section from interface 400F may not shift in interface 400G, and thereby the contents in this highlighting bracket are still assessed. Control unit 103 can determine if the criterion used to make an assessment have been modified. In response to determining the highlighting brackets have shifted in positions, the criterion has been changed and the veterinarian is prompted to modify to the assessment of the pet. In this embodiment, the assessment changes from A to B, based on the new criteria. Additionally, there are buttons on the bottom of interface 400G, which can be pressed to: (i) close out interface 400G via an exit button; (ii) save modifications to interface 400G via a save button; and (iii) proceed to publish the reports with the sections mentioned above via an approve button. The brackets which appear in interface 400F are absent in the published report, which can be in PDF form.

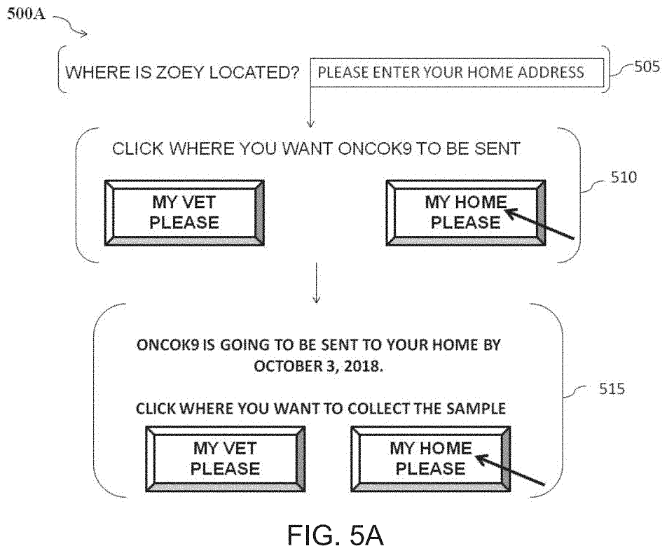

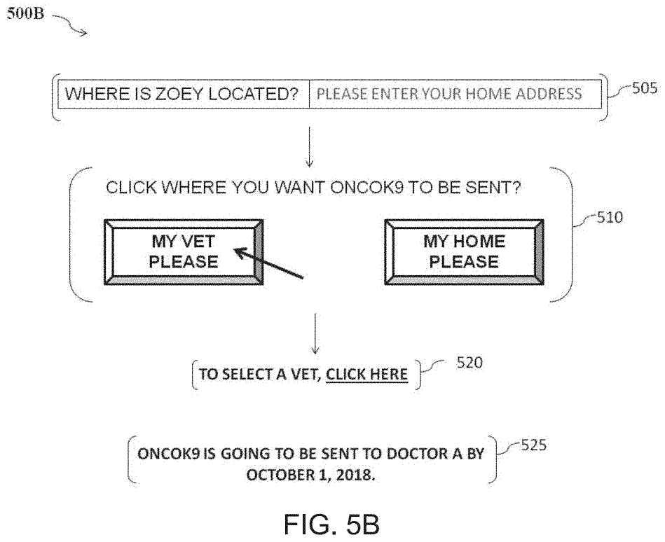

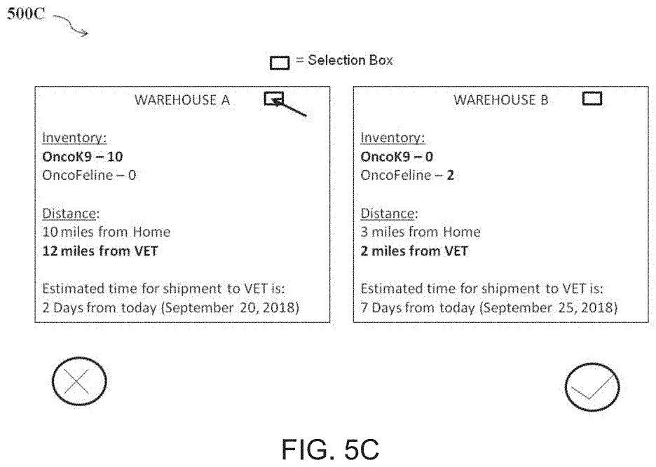

Referring to FIG. 5A, an interface 500A supports a direct-to-consumer (DTC) setup where the consumer entity only engages with control unit 103 in server 105. In an embodiment, Zoey is a pet of the consumer entity and OncoK9™ is the selected medical test. In prompt 505, the consumer entity is asked to enter in location (i.e., home address of the consumer entity) of the pet. In response to entering in the information for prompt 505, control unit 103 sends interface 510, which asks the consumer entity where OncoK9™ should be sent. There are two buttons in interface 510—“MY VET PLEASE” for sending the medical testing kit for OncoK9™ to the clinical entity and “MY HOME PLEASE” for sending the medical testing kit for OncoK9™ to the home of the consumer entity.

In response to clicking on the “MY HOME PLEASE” button, control unit 103 can coordinate with the logistics entity (as described above) to send OncoK9™ to the home of the consumer entity, as depicted in interface 515. There are two buttons in interface 510—“MY VET PLEASE” for collecting the sample for the medical testing kit for OncoK9™ to the clinical entity and “MY HOME PLEASE” for collecting the sample for the medical testing kit for OncoK9™ to the home of the consumer entity. In an embodiment, if “MY VET PLEASE” button is selected, then interfaces which correspond with the operations in flowchart 300 (as described above) are presented to consumer entity. In another embodiment, if “MY HOME PLEASE” button is selected, then control unit 103 in server 105 connects to database 120. The contents of database 120 can also include information and availability of mobile phlebotomists. Control unit 103 can generate a list of phlebotomists (not depicted) and their availability that similar to interface 245 and interface 250, respectively. This removes the need for: (i) the consumer entity to go to the clinical entity to collect a sample for the medical testing kit; and (ii) shipping the sample to the testing entity from the clinical entity. The mobile phlebotomists can transport or organize the shipping for getting the sample to the testing entity. Additionally, this would be a convenience for consumer entities residing in rural and isolated areas or if the consumer entity or pet are not healthy enough for travel to the clinical entity.

Referring to FIG. 5B, the consumer entity is asked to enter in location (i.e., home address of the consumer entity) of the pet in prompt 505. In an embodiment, control unit 103 sends interface 510, which asks the consumer entity where OncoK9™ should be sent in response to receiving the information for prompt 505. There are two buttons in interface 510—“MY VET PLEASE” for sending the medical testing kit for OncoK9™ to the clinical entity and “MY HOME PLEASE” for sending the medical testing kit for OncoK9™ to the home of the consumer entity. In response to clicking on the “MY VET PLEASE” button, control unit 103 can coordinate with the logistics entity (as described above) to send OncoK9™ to the office of the clinical entity by clicking on the underlined link, as depicted in interface 520. More specifically, by clicking on the underlined link, control unit 103 in server 105 sends interfaces 245 (i.e., the list of veterinarians) and 250 (i.e., days the sample can be sent to the selected veterinarians among the clinical entities) to the consumer entity. In response to selecting the interfaces 245 and 250, a message 525 is sent to the consumer entity by control unit 103. Message 525 states OncoK9™ is going to be sent to selected veterinarian, Doctor A, by the date of Oct. 1, 2018.

Referring to FIG. 5C, control unit 103 in server 105 can be integrated with the enterprise resource planning (ERP) systems of warehouses or other entities which have medical testing kits of interest to different consumer entities. The logistics entity can transport the medical testing kit directly to the consumer entity or clinical testing entity. An interface 500C may result, as depicted, from control unit 103 connecting ERP systems of each warehouse. In an embodiment, control unit 103 connects warehouse A and warehouse B, which have inventories of OncoK9™ and OncoFeline™. In this embodiment, the consumer entity wants to order and send the medical testing kit of OncoK9™ to his or her selected veterinarian (i.e., selected clinical entity). The selected medical testing kit, which is OncoK9™, becomes bolded for warehouse A and warehouse B. Additionally, the distance of the selected destination for OncoK9™, which is the selected clinical entity, becomes bolded for warehouse A and warehouse B. Based on the inventory and distance and coordinating with the logistic entity, control unit 103 determines an estimated shipping time to the destination, which is the VET (i.e., the clinical entity) from each warehouse. The consumer entity can select warehouse A or warehouse B by clicking on a selection box. If the consumer entity consumer entity clicks on the “x” in the bottom left hand corner of interface 500C, then the consumer entity exits out of interface 500C. If the consumer entity clicks on the “✓” in the bottom right hand corner of interface 500C, then control unit 103 connects to program 107 in logistics device 135 to coordinate the shipment of the OncoK9™ to desired destination—the home of the consumer entity or the office of the clinical entity.

FIG. 6A shows one embodiment of a computing environment 600A, that includes a control unit 103 in server 105 and applies filter processes, AI techniques, and data analytics. Control unit 103 may apply filter process 605 on the contents of database. Database 120 may contain clinical entities and testing entities that have an arrangement with the coordination entity. Arrangements can be based on AI techniques which determine the clinical entities and testing entities, best suited for the medical testing preferred by control unit 103 operated by the coordination entity. List 610 and list 620 are filtered-in lists of clinical entities and testing entities suited for the medical testing preferred by the coordination entity. List 615 and list 620 are filtered-out lists of clinical entities and testing test not as well suited for the medical testing preferred by the coordination entity. Control unit 103 applies AI techniques 630 on list 610 and list 620 to generate resultant 635. In resultant 635, control unit 103 determines which logistic entities are best suited for delivering: (i) the medical testing kit containing the sample to the consumer entity; and (ii) the sample from clinical entities to testing entities.

As shown in FIG. 6B, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical applications, or technical improvement over technologies found in the marketplace, or to enable others of ordinary skill in the art to understand the embodiments disclosed herein.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description, which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A computer implemented method for assessing a health state of canines or other animals, the computer implemented method comprising:
receiving an order from an individual consumer for a medical test without the need for a prescription and thereby shipping a medical testing kit to the individual consumer or a clinician or the individual consumer and the clinician, wherein the medical testing kit contains one or more items for supporting the medical test;
confirming receipt of the medical testing kit by the individual consumer;
sending instructions to collect a sample for the medical testing kit;
shipping the medical testing kit containing the sample to a medical testing laboratory, wherein the sample is stored in an item of the one or more items and the sample derives from the canines or other animals;
confirming receipt of the sample in the medical testing kit by the medical testing laboratory;
making an assessment of the health state based on criteria applied on the sample in the medical test kit; and
sending a prompt to a clinician, wherein the prompt triggers an interface for optionally modifying the assessment.

2. The computer implemented method of claim 1, further comprising: receiving a profile of the sample.

3. The computer implemented method of claim 1, wherein shipping the medical testing kit containing the sample comprises: receiving a verification of the profile of the sample prior to shipping the sample by the clinician.

4. The computer implemented method of claim 2, wherein the profile is processed electronically.

5. The computer implemented method of claim 1, wherein the medical test is a liquid biopsy test for detection of cancer.

6. The computer implemented method of claim 1, wherein the assessment of the health state is sent to a GUI by the clinician prior to sending the assessment of the health state to the individual consumer.

7. The computer implemented method of claim 1, wherein the sample is blood.

8. The computer implemented method of claim 7, wherein the medical testing kit contains a blood tube designed to stabilize ctDNA for liquid biopsy applications.

9. The computer implemented method of claim 1, further comprises: generating a list sent to the individual consumer, wherein the list contains clinicians for selection by the individual consumer, and wherein a clinician selected from the list collects the sample for the medical testing kit and places the sample in the medical testing kit.

10. The computer implemented method of claim 1, further comprising: sending the assessment of the health state to the individual consumer or to a GUI as a PDF.

11. A computer implemented system for ordering a medical test for canines or other animals by an individual consumer, the computer implemented system comprising:
a server;
a computing device in communication with the server over a network, the computing device includes a memory coupled to the processor and configured to provide the processor with instructions for:
receiving order directly from the individual consumer without the need for a prescription for the medical test and thereby shipping the medical testing kit to the individual consumer or a clinician or the individual consumer and the clinician, wherein the medical testing kit contains one or more items for supporting the medical test;
sending a request to a logistics organization to ship the medical kit to the individual consumer or the clinician;
receiving a confirmation of receipt that the individual consumer received the medical kit;
receiving a request from the individual consumer to select the clinician to collect a sample for the medical testing kit;
sending a notification of receipt of the sample by a testing laboratory to the individual consumer, wherein the sample is stored in an item of the one or more times and the sample derives from the canines or other animals;
sending a notification of the availability of a diagnosis obtained from the medical test of the sample to the individual consumer;
making an assessment of a health state of the canines or other animals, based on criteria applied on the sample in the medical testing kit;
sending a notification of the assessment of the health state to the individual consumer; and
sending a prompt to the clinician, wherein the prompt triggers an interface for optionally modifying the assessment.

12. The computer implemented system of claim 11, wherein the medical test is a liquid biopsy test for the detection of cancer.

13. The computer implemented system of claim 11, further comprising instructions for:
generating a list sent to the individual consumer, wherein the list contains clinicians for selection by the individual consumer, and wherein a clinician selected from the list collects the sample for the medical testing kit and places the sample in the medical testing kit;
receiving a request from the individual consumer to schedule an appointment with the selected clinician; and
sending a notification of confirmation that the appointment has been scheduled with the selected clinician.

14. The computer implemented system of claim 11, wherein the sample is blood.

15. The computer implemented system of claim 11, wherein the sample test kit contains a blood tube designed to stabilize ctDNA for liquid biopsy applications.

16. The computer implemented system of claim 11, further comprising instructions for: receiving a profile of the sample.

17. A computer program product for ordering a medical test for canines or other animals by an individual consumer, the computer program product comprising:
a server;
a computing device in communication with the server over a network, the computing device includes a memory coupled to a processor and configured to provide the processor with instructions for:
receiving an order directly from the individual consumer without a prescription for the medical test and thereby shipping the medical testing kit to the individual consumer or a clinician or the individual consumer and the clinician, wherein the medical testing kit wherein the medical testing kit contains one or more items for supporting the medical test;
sending a request to a logistics organization to ship the medical testing kit to the individual consumer;
receiving a confirmation of receipt that the individual consumer received the medical testing kit;
receiving a request from the individual consumer to select the clinician to collect a sample for the medical testing kit, wherein the sample is stored in an item of the one or more times and the sample derives from the canines or other animals;
sending a notification of receipt of the sample by a testing laboratory to the individual consumer;
sending a notification of the availability of a diagnosis obtained from the medical test of the sample to the individual consumer;
making an assessment of a health state of the canines or other animals, based on criteria applied on the sample in the medical testing kit;
sending a notification of the assessment of the health state to the individual consumer; and
sending a prompt to the clinician, wherein the prompt triggers an interface for modifying the assessment.

18. The computer program product of claim 17, wherein the medical test is a liquid biopsy test for the detection of cancer.

19. The computer program product of claim 17, further comprising
generating a list sent to the individual consumer, wherein the list contains clinicians for selection by the individual consumer, and wherein a clinician selected from the list collects the sample for the medical testing kit and places the sample in the medical testing kit;
receiving a request from the individual consumer to schedule an appointment with the selected clinician, and
sending a notification of confirmation that the appointment has been scheduled with the selected clinician.

20. The computer program product of claim 17, wherein the sample is blood.

21. The computer program product of claim 17, wherein the sample test kit contains a blood tube designed to stabilize ctDNA for liquid biopsy applications.

22. The computer program product of claim 17, further comprising: receiving a profile of the sample.
